(19) United States
(12) Patent Application Publication
(10) Pub. No.: US 2024/0350169 A1
(43) Pub. Date: Oct. 24, 2024

(54) CANNULA ASSEMBLY SYSTEMS AND METHODS

(71) Applicant: Revelle Aesthetics, Inc., Mountain View, CA (US)

(72) Inventors: Jonathan Podmore, San Carlos, CA (US); Earl Bright, II, Sunnyvale, CA (US); Arthur Ferdinand, Gilroy, CA (US); John Hanley, Manhattan Beach, CA (US)

(21) Appl. No.: 18/638,080

(22) Filed: Apr. 17, 2024

Related U.S. Application Data
(60) Provisional application No. 63/460,215, filed on Apr. 18, 2023.

(51) Int. Cl. A61B 17/34; A61B 17/00; A61M 1/00

(52) U.S. Cl. CPC A61B 17/3403; A61B 17/3421; A61M 1/895; A61B 2017/00128; A61B 2017/3413

(57) ABSTRACT

Systems and methods for fat injection or liposuction including or involving a cannula that is configured to provide feedback concerning depth within tissue or the environment into which the cannula is placed.

### FIELD OF THE DISCLOSURE

The present disclosure generally relates to systems and methods for aesthetic treatments of the body.

### BACKGROUND OF THE DISCLOSURE

There is a continuing need for safe and effective approaches to fat injection or liposuction. Such procedures have become common and more readily performed as aesthetic procedures on the body.

Fat injection procedures can be performed throughout the body and there is associated therewith varying degrees of complexity and risk. One procedure commonly known as a Brazilian Butt Lift (BBL) is among the riskier aesthetic procedures. In fact, the published mortality rate associated with BBL procedures is 1:3000 deaths. Consequently, there is a need to make such procedures more safe.

The goal of fat injection or fat grafting procedures is to take volume from one area of the body and add volume to certain areas of the body. With BBL procedures, volume is added to the buttocks. However, the risk associated with these and related procedures is increased due to the possibility of a fat embolism with the injected fat finding its way to the bloodstream which can lead to procedure complications, including death.

A number of investigations have been conducted to determine proper approaches for safe fat injection. One approach to improve safety is to know the depth at which the tip of the fat injection cannula is being employed. In this way, risks associated with fat injection procedures can be minimized.

However, conventional fat injection is often limited by the devices employed in an injection procedure. Such devices generally are not capable of providing feedback concerning the depth or the environment into which the cannula is placed.

Information concerning the depth and environment into which a cannula is placed is also useful to liposuction and cellulite treatment procedures where such procedures are conducted alone or in combination with fat injection. In liposuction procedures, such a cannula can be employed to better navigate to and target the best areas for removing fat. For cellulite treatment procedures involving engaging or cutting septa, such a cannula can be used to navigate about and engage target septa.

Accordingly, there is a need for an improved approach to a cannula for fat injection or collection, cellulite treatment tools, or other devices and methods. Such approaches should be associated with predictable and safe results and be relatively easy to employ.

The present disclosure addresses these and other needs.

### SUMMARY OF THE DISCLOSURE

Briefly and in general terms, the present disclosure is directed towards systems, devices and methods for performing fat injection and/or liposuction, and cellulite treatment. In various approaches, there is provided an aesthetic treatment system including a cannula assembly embodying structure and functionality to provide device depth, relative positioning within tissue, and/or tissue environment information.

In one aspect, the treatment system provides a user with feedback regarding the depth that the cannula is placed within tissue or additionally, information regarding relative positioning or the environment into which the device is placed. In this way, fat insertion, fat grafting or liposuction can be more safely performed and more considered decisions regarding treatment can be made. Specific tissues or anatomy can be targeted and/or avoided as desired. In another aspect, structure providing such feedback information is embodied in a tool that can be added to a standard fat grafting or liposuction cannula. In one approach, such structure defines a sheath or sleeve that is placed over or otherwise defines structure that engages the standard treatment apparatus. In another approach, the feedback providing structure is fed into a lumen of the standard apparatus.

In one particular approach, ultrasound is employed for tissue depth sensing. An ultrasound transducer is positioned at the tip of the cannula and a simple scan is conducted. Ultrasound energy is transmitted to reflect off of a skin/air interface which results in a strong signal returning to the transducer. This reflection of energy is analyzed to determine tool depth. In this way, a user can determine where and/or the environment in which fat is being delivered, or removed, in an aesthetic treatment procedure.

The cannula assembly can also involve in certain approaches, illumination such as a light configured at or emitted through a tip of cannula structure or placed along or at strategic locations along the cannula for the purposes of tracking advancement of the tool to the treatment site and locating intra-dermal structures at the treatment site. In this way, direct observation of the treatment device by transillumination through the skin is provided and positioning and performance thereof subcutaneously is readily available to an operator. Moreover, the light is configured to be visible at depths where fat grafting or removal is acceptable and generally considered safe. At deeper depths, the light would fade and communicate to the user that the cannula depth is too deep.

These and other features of the disclosure will become apparent to those persons skilled in the art upon reading the details of the systems and methods as more fully described below.

### BRIEF DESCRIPTION OF THE DRAWINGS

FIG. 1A is a side view, depicting a cannula assembly located subcutaneously within tissue and generating ultrasound energy.

FIG. 1B is a side view, depicting the cannula assembly of FIG. 1A receiving reflected ultrasound energy.

FIG. 1C is a side view, depicting a cannula assembly generating and receiving ultrasound energy and emitting light energy.

FIG. 1D is a side view, depicting an alternate approach to structure configured to facilitate assessing depth of a cannula assembly.

FIGS. 2A-D are perspective and side views, depicting various alternative approaches to a cannula assembly including light emitting structure.

FIG. 3 are side views, depicting use of a cannula assembly in assessing tissue depths and positioning relative to tissue structures.

FIGS. 4A-D are front and perspective views, depicting various approaches to displaying depth or cannula positioning information.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

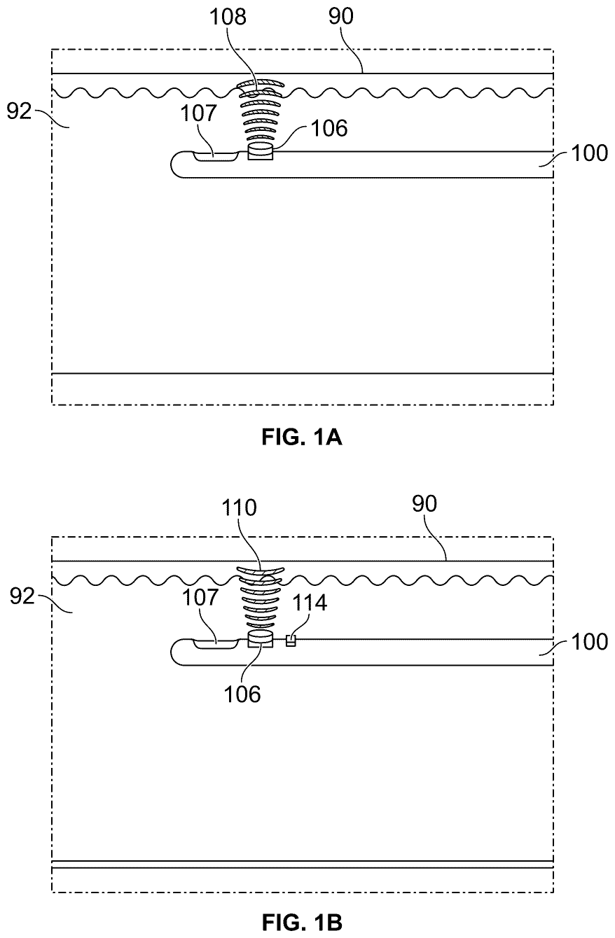

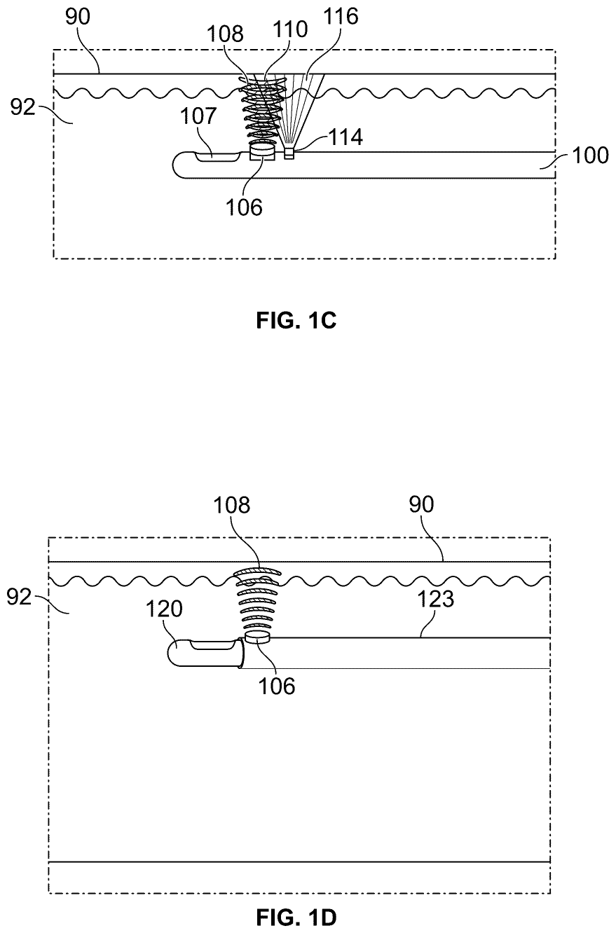

Referring to FIGS. 1A-B, there is shown a generally elongate cannula device or assembly 100 that forms part of an aesthetic treatment system and which is equipped with an integrated ultrasound sensor or transducer 106 configured near a distal end portion of the cannula device 100.

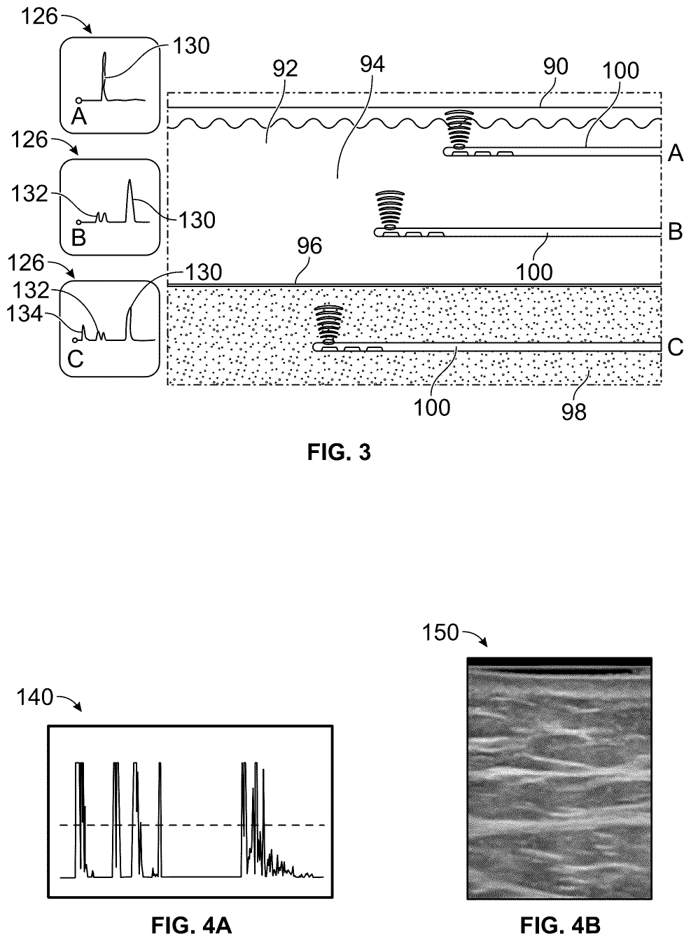

The cannula device 100 includes or communicates with a controller that processes information resulting from or associated with the emission and receipt of ultrasound energy generated by the transducer 106. In one aspect, the controller includes a program specific to handle ultrasound energy information used in conjunction with the cannula device 100 and comprises or includes a non-transitory computer-readable storage medium and a computer-program mechanism embedded therein to process ultrasound energy-based information. Accordingly, either a direct electrical connection or telemetry is employed to provide communication between the device 100 and the controller (not shown). A port or window 107 is formed in the cannula 100 just distal to the ultrasound sensor 106, but can be located at other positions along its length, and also additional windows can be formed in the cannula 100 (See also FIG. 3). Moreover, in various embodiments, the window 107 is configured on an opposite side of the cannula 100 relative to sensor structure or the cannula 100 can be rotated axially relative to the sensor structure so that the user can position the windows 107 as desired relative to the sensor structure. It is through this window 107 that fat or other biological material can be collected or deposited as desired. In this specific regard, a proximal end (not shown) of the device 100 is provided with functionality to accomplish advancing collected fat out of the window 107, or to collect fat through the window 107 as is known in the art.

As shown in FIG. 1A, the cannula 100 is positioned subcutaneously below the skin 90 in the fat 92. The ultrasound sensor 106 is configured to send an ultrasound signal 108 that is analyzed or manipulated by the controller to facilitate assessing tip depth. Reflected ultrasound signal energy 110 is received by the sensor 106 as shown in FIG. 1B. A LED 114 or other light source, such as a fiber optic conveying light from a proximal end, can additionally be positioned near the ultrasound sensor 106 and configured to aid in tip tracking.

Using A-mode ultrasound, a simple scan can be conducted by the cannula device 100. With the cannula device 100 in the space of the target area, the interface between the surface of the transducer 106 and tissue should be ideal and air free. With the signal generation utilized to assess the tissue, ultrasound near perfectly reflects off of the skin/air interface, thus resulting in a signal returning to the transducer 106 that represents the distance from the cannula to the skin surface. Other structures, while reflective of ultrasound, do not return an amplitude of signal commensurate with this interface. In this way, the positioning of the cannula device 100 relative to fat deposits can be ascertained with respect to the surface of skin by analyzing the generation and collection of ultrasound energy.

In one approach, the cannula device 100 can have a 3-4 mm diameter, but larger and smaller cannulas are contemplated such as ranging from 0.5-5 mm or more. It is noted that either vibratory cannulas or non-powered cannulas can be employed in a treatment procedure. Moreover, the cannula device 100 can be a standalone apparatus with built-in sensors. Depending on cost and implementation, the cannula device 100 or portions thereof can be disposable, reusable, or limited reusable devices.

The cannula device 100 thus provides structure and functionality lending itself to use in both fat injection and liposuction procedures by providing necessary feedback to the user concerning device depth, or its relative position to various tissues. In an alternative or additional embodiment, the cannula device 100 can also provide feedback concerning the environment into which the device is placed. In this way, various tissues and structures can be selectively targeted, avoided or navigated about.

The cannula 100 can be a liposuction cannula or fat injection cannula that also involves in certain alternative or additional approaches, illumination such as a bright light configured at or emitted through a tip of cannula structure or placed along or at strategic locations along the cannula for the purposes of tracking advancement of the tool to the treatment site and locating intra-dermal structures at the treatment site. In this way, direct observation of the treatment device 100 by transillumination through the skin is provided and positioning and performance thereof subcutaneously is readily available to an operator. Moreover, the light is visible at depths where fat grafting is acceptable and generally considered safe. At deeper depths, the light would fade and communicate to the user that the cannula depth is too deep.

In one approach, a Red, Green, Blue LED for transillumination and tip tracking can be incorporated into the cannula 100 where the color of the LED is changed in accordance with the sensed information. This allows the user to keep eyes on the patient and device, as opposed to having to watch a monitor during various stages of device use. In yet other embodiments, the cannula 100 can be equipped with two fixed wavelength LED's which through switching relay feedback to the user, or the cannula 100 can be configured to use a single LED which either is turned on or off or flashes in response to sensor feedback. Moreover, the cannula 100 can include a light source that has light output or intensity levels that are preset, or that is user set so that adjustments can be made during a procedure based upon patient skin type or is supported with secondary ultrasound imaging.

With reference to FIG. 1C, the sensor 106 and LED 114 are shown in operation. The sensor 106 is both sending out ultrasound energy 108 and receiving reflected ultrasound energy 110. Additionally, the LED 114 is emitting light energy 116 that can be used in a transillumination localization procedure. Furthermore, to provide an ultrasound image of tissue existing in the environment into which the device is placed, the cannula device 100 can be equipped with a more complex transducer and drive system.

In an alternative embodiment (FIG. 1D), the depth and localization functionality can be added to a standard fat grafting or liposuction cannula 120. In one approach, a sheath or sleeve 123 embodying such ultrasound functionality can be configured over a standard cannula 120. This allows the user to continue to use tools they are familiar with while adding desirable functionality. The sleeve 123 is configured to allow for some variation in cannula size and desired sensor locations and be designed to be reusable or disposable. Additionally, it takes into account various distinct types of cannulas to both allow sleeve 123 placement or adjustment. In various embodiments, the sleeve 130 can be tube-like and slide over standard cannulas or they can have a longitudinally extending expansion zone (not shown) which would accommodate some variability in cannula size. Also, while providing structure that creates a smooth transition between the standard cannula and the sleeve 123, securing structure can also be provided to make sure the sleeve 123 travels with the standard cannula during treatment procedures.

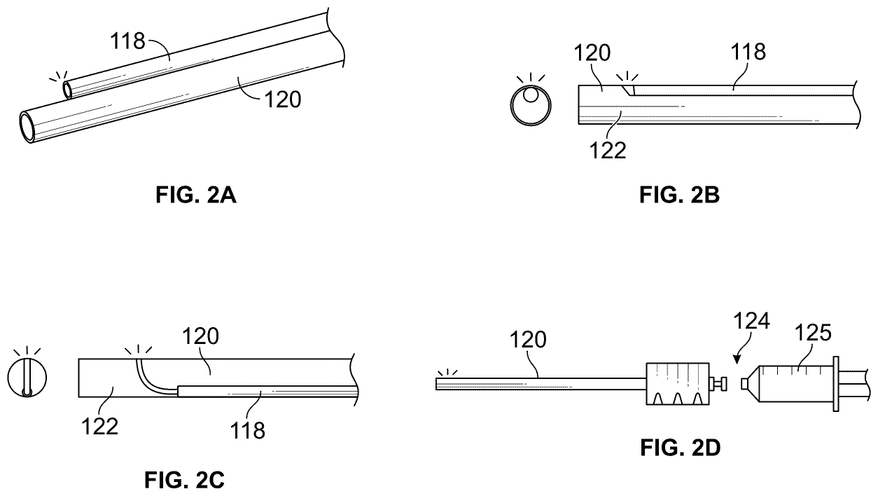

Alternatively, structure including light generating functionality 118 can be fed along the outside of a standard cannula 120 (FIG. 2A) or within an internal lumen 122 of the standard cannula 120 (FIGS. 2B-C). Further, the assembly can include a luer connection 124 at a proximal end thereof that allows for fiber and light source to exit off to the side and allow for fat in syringe 125 to still be operatively attached to the cannula 120 (FIG. 2D). In this way, the user can still use the standard cannula 120 and also be provided with means to assess positioning, depth and the environment surrounding the cannula 120.

Using A-mode ultrasound, as stated, a simple scan can be conducted by the cannula device 100. With this reflection information and by understanding the phase of the received signal along with the speed of sound in tissue (typically 1540 m/s), an assessment of the depth of the cannula device 100 can be made. One goal of an A-scan ultrasound approach is to be able to use a simplified, potentially even single piece ultrasound transducer in an effort to create an economical device. With a single piece transducer, an A-scan can return to the user information about the tissue in which the cannula device 100 is placed.

In the subcutaneous space, there are a number of pieces of information that could be assessed with an A-scan 126 (See FIG. 3). First, device depth A from the skin surface 90 can be assessed as skin provides a near perfect reflector of an ultrasound signal. For example, peak 130 represents the depth at device position A. Additionally, at device depth B, an A-scan 126 can be utilized to provide information concerning the location of a treatment cannula with respect to superficial facia 94 of interest, or example, peak 132 on an A-scan can represent the location of superficial fascia 94 in the buttock, for example at a depth B approximately 10-12 mm from the skin surface. When the device is placed too deep for safe fat injection such as device depth C, the location of deep fascia 96 attached to the gluteal muscle 98 (depth dependent on the thickness of fat that is present in the deep fat compartment) for example at a depth C potentially ranging from 20-40 mm from the skin surface is represented by A-scan peak 134.

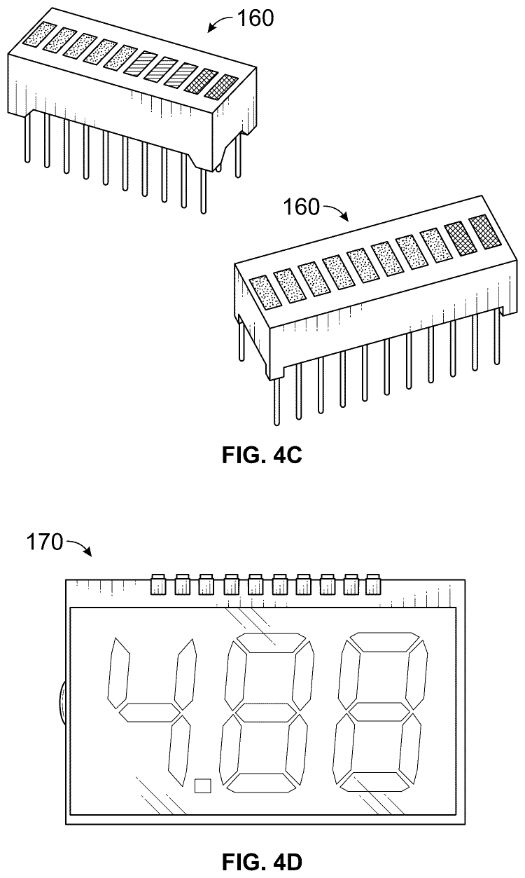

Moreover, the system controller can be configured to provide various approaches to data presentation to a user for assessment and action (See FIGS. 4A-D). Approaches can range from simple A-scans 140 presented on a display, monitor or tablet (FIG. 4A) to highly comprehensive such that a full ultrasonic image 150 from the cannula tip to the skin surface is presented (FIG. 4B). To provide a full ultrasonic image, a multi-segment transducer that facilitates creating a desired and detailed image is employed.

In another approach, processing of the received signal by the system controller can be conducted to automatically determine depths and either directly relay it to the user on a screen or with prescribed feedback. For example, a green light can be caused to be illuminated by the controller on the device 100 when depths are within a predetermined range. In particular, where an LED is employed on the device 100, a flash or color change could notify the user of acceptable or unacceptable depths 160 (See FIG. 4C). A pure digital measurement of depth or depth from a target such as the superficial fascia also could be displayed 170 to a user (FIG. 4D).

In alternative embodiments, other sensor technologies (thermal, capacitance, optical), can be applied to the end of a cannula device 100 to return depth or environment information. Additionally, complementary devices or structures can be placed on the skin surface to facilitate measuring capacitance between the treatment cannula and skin. In this specific regard, a sheet of metal can be placed on the skin above a treatment site or a known strength magnet with a sensor can be configured on the cannula in order to assess magnetic strength to arrive at tissue depth.

Additionally, or alternatively, in each disclosed embodiment, illumination can be employed and provided via a lightguide from an external light source or via one or more LEDs external or internal the treatment device. Illumination aids the user both with locating the treatment device as well as proper depth placement as transillumination decreases with increasing tool depth. In one aspect, the amount of illumination is set to ensure proper depth of a treatment device or structure, the level of illumination targeted being adjusted for skin type, thickness, presence of fat and pigment.

Treatment plans or paths can be generated automatically by employing a computerized controller programmed to most efficiently address and measure a pre-defined treatment site. The computerized controller can be associated with a scanner that identifies specific areas for treatment. In this regard, the computerized controller includes a program specific to fat insertion or liposuction and is used in conjunction with an electronic and mechanical device and comprises or includes a non-transitory computer-readable storage medium and a computer-program mechanism embedded therein to both identify treatment areas and to plot primary and alternative approaches to treatments. In another embodiment, computerized visualization and treatment planning equipment is used to assist the physician in determining insertion site locations and paths to be taken to the marked targets.

In one specific additional embodiment, a measurement device can be employed to create a complete three-dimensional map of all target treatment areas. By dating and comparing treatment areas versus normal idealized surfaces, the operator calculates total and local volume benefits of therapy and track improvement over time.

After completing treatment of one target area, the procedure is repeated to treat other target areas. Accordingly, the same device can be employed to access tissue layers at or below other fat injection or liposuction sites. It is to be recognized that the system can further include structure permitting the assembly to be steerable to subcutaneous treatment sites. In such an embodiment, the device would be configured to define longitudinally flexible material, and the instrumentation would be steered to the desired position within tissue. Moreover, in certain applications, the device has a stiffness that varies along its length. In another embodiment, the treatment device is embodied in a deflectable cannula.

Accordingly, various approaches to tissue treatment methods and apparatus are presented. The disclosed approaches are configured to provide an effective and focused approach to smoothing tissue and treating, minimizing and preventing cellulite. The disclosed approaches can also be used to repair and improve the appearance of tissue in a targeted manner. Further, the disclosed proactive treatment modalities are easy and effective to use.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. An aesthetic treatment system, comprising:
a cannula assembly defining an elongate profile and having a proximal end portion and
a distal end portion, the cannula assembly being configured to inject or collect fat; and
an ultrasound transducer configured at the distal end portion.

2. The aesthetic treatment system of claim 1 further comprising:
a light emitter configured at the distal end portion.

3. The aesthetic treatment system of claim 1, wherein the ultrasound transducer is configured to send and receive an ultrasound energy, wherein the system is configured to analyze the ultrasound energy to assess a depth of the cannula assembly within tissue.

4. The aesthetic treatment system of claim 2, wherein the light emitter is configured to provide transillumination for tracking the cannula assembly within tissue.

5. The aesthetic treatment system of claim 2, wherein the light emitter is configured to provide depth information of the cannula assembly within tissue.

6. The aesthetic treatment system of claim 1, further comprising a controller configured to process ultrasound energy information.

7. The aesthetic treatment system of claim 1, further comprising a controller configured to process light energy information.

8. The aesthetic treatment system of claim 1, further comprising a display, wherein the controller is configured to process and present ultrasound energy information or light energy information on the display.

9. The aesthetic treatment system of claim 1, wherein A-scans are presented on a display.

10. The aesthetic treatment system of claim 1, wherein a highly comprehensive or a full ultrasonic image is presented on a display.

11. The aesthetic treatment system of claim 1, wherein a digital display is employed to provide depth information.

12. The aesthetic treatment system of claim 1, further comprising an LED that flashes or changes color to represent a depth of the cannula assembly.

13. The aesthetic treatment system of claim 1, further comprising a standard cannula and a sleeve including depth assessment structure and functionality and being sized and shaped to engage and attach to the standard cannula.

14. The aesthetic treatment system of claim 1, wherein the cannula assembly is steerable.

15. The aesthetic treatment system of claim 1, wherein the cannula assembly is longitudinally flexible.
